(19) United States
(12) Plant Patent Application Publication
(10) Pub. No.: US 2006/0015976 P1
(43) Pub. Date: Jan. 19, 2006

(54) ACER SHIRASAWANUM PLANT NAMED 'MUNN 001'

(50) ACER SHIRASAWANUM; ACER

(76) Inventor: Carl A. Munn, Brooks, OR (US)

Correspondence Address: KLARQUIST SPARKMAN, LLP, 121 SW SALMON STREET, SUITE 1600, PORTLAND, OR, 97204, US

(21) Appl. No.: 10/895,191

(22) Filed: Jul. 19, 2004

(51) Int. Cl. A01H 5/00

(52) U.S. Cl. PLT/216

(57) ABSTRACT

A new and distinct cultivar of Acer shirasawanum plant as shown and described, characterized by its striking carmine to red spring leaves, yellow to lime summer leaves, and vigorous growth rate.

### BOTANICAL CLASSIFICATION

Acer shirasawanum

### VARIETY NOMINATION

‘MUNN 001’

The present invention relates to a new and distinct cultivar of deciduous tree of the genus and species, Acer shirasawanum, and given the name ‘MUNN 001’. The genus Acer is of the family Aceraceae.

The new cultivar was found in a cultivated area by Carl Munn as a chance seedling of Acer shirasawanum ‘Aureum’, an unpatented plant.

Acer shirasawanum ‘Aureum’ is described in the A to Z Encyclopedia of Garden Plants as a “Rounded, bushy, deciduous tree or shrub producing rounded, 7 to 11-lobed, bright yellow leaves, 7-10 cm long. Tiny, red-purple flowers are borne in upright corymbs.” Acer shirasawanum ‘Aureum’ is a problem for growers due to its slow growth. It is also difficult to propagate as it doesn't take a graft well. In addition, its leaves burn badly in full sun.

This new cultivar is characterized by the following:

1. Vigorous and fast and upright growing;

2. Carmine to red new growth in spring fading to gold in summer;

3. Sun tolerance, can be grown in full sun with only light burning on top leaves;

4. Lime green foliage where shaded.

Asexual propagation by side graphs in Brooks, Oreg., shows that the foregoing characteristics and distinctions come true to form and are established and transmitted through succeeding propagations. The present invention has not been evaluated under all possible environmental conditions. The phenotype may vary with changes in environment without a change in the genotype of the plant.

### BRIEF DESCRIPTION OF THE DRAWING

FIG. 1 shows mature 17 year old plant growing in the ground in Brooks, Oreg.

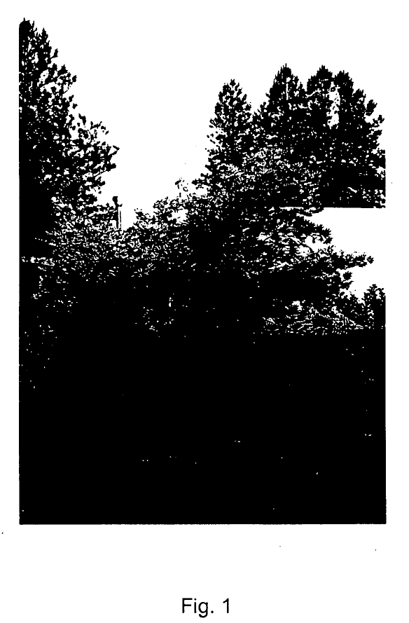

FIG. 2 shows a close up of a summer branch and foliage.

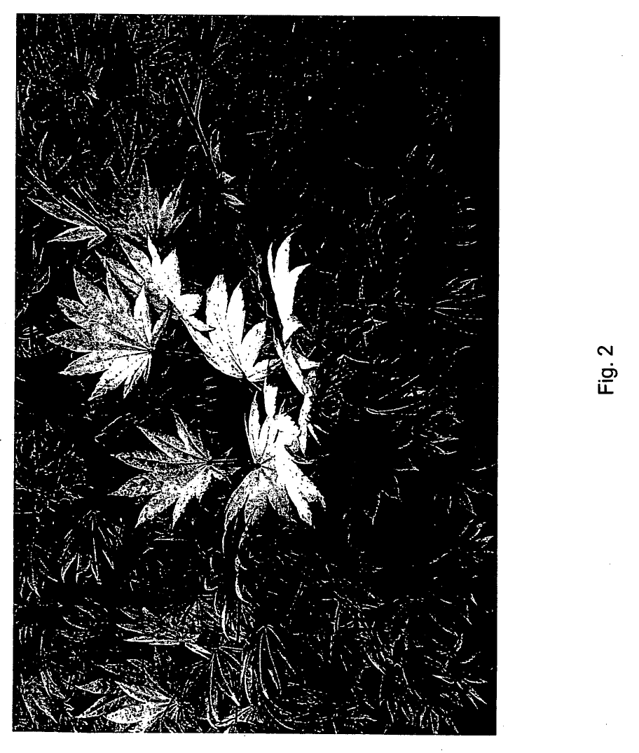

FIG. 3 shows the spring leaf color.

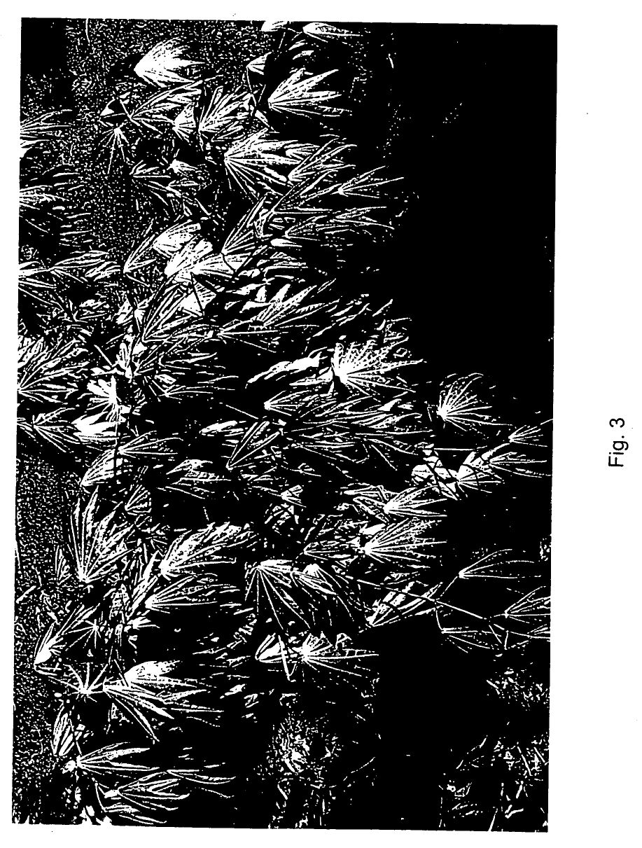

FIG. 4 shows a close up of the flowers.

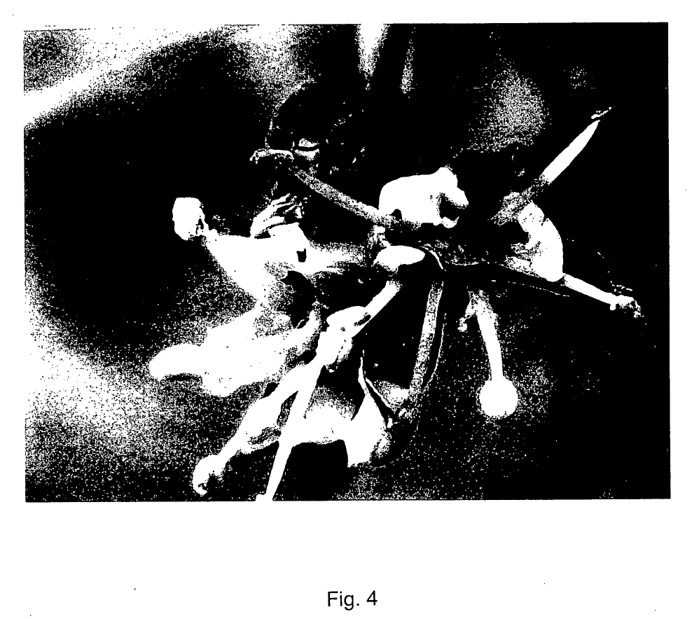

### DETAILED PLANT DESCRIPTION

The following is a detailed description of the new cultivar based on observations taken in early July of 2004 of a four-year-old specimen grown in the ground in full sun in Brooks, Oreg. The flower and fruit data are from branches of a 17 year old plant grown in the same manner. The color descriptions are all based on The Royal Horticultural Society Colour Chart.
Plant:
   Type.—Deciduous tree.
   Hardiness.—USDA Zones 5 to 7.
   Size.—Mature size unknown (17 year old tree has grown to about 4 meters tall and 3.5 meters wide).
   Form.—Globose.
   Growth.—Vigorous, typical four year old plants grown in Brooks, Oreg., exhibited about 90 cm growth in the leader and primary side branches during the 2004 growing season through the 1^{st} of July 2004.
Stem:
   Type.—Woody.
   Size.—Trunk base of four year old specimens had a caliper that is 2.3 cm to 3.2 cm (caliper measured one inch above ground).
   Surface.—Young stems are glabrous. Two year old stems have 0.5 mm lenticels. Lenticels increase in size with age.
   Color.—This years' growth is Greyed-Orange 175A in the sun and Yellow-Green 146B in the shade. Last years' growth is Yellow-Green 146B with lenticels of Brown 199B to Grey 198D. Three year old stems are striated Brown 199B and Grey 198D. The trunk bark color is closest to 197A.
Leaf:
   Type.—Simple.
   Shape.—Orbicular.
   Arrangement.—Opposite.
   Lobing.—7 to 11, parted down one-third to one-half to the leaf base. Lobes ovate, attenuate.
   Margins.—Doubly serrate.
   Apex.—Attenuate.
   Base.—Cordate.
   Venation.—Palmate.
   Length.—Grows to 10 cm.
   Width.—Grows to 10 cm.
   Vestiture.—Glabrous (just unfolding leaves pubescent).
   Surface.—Matte.
   Texture.—Papery.
   Petiole length.—3.5 to 5.5 cm.
   Petiole texture.—Glabrous.
   Petiole color.—Greyed-Purple 185A on new leaves to Greyed-Red 178A on older leaves.
   Color.—Topside — Emerges a brilliant carmine, Red-Purple 64B which darkens to Greyed-Red 178A with yellow green veins, Yellow-Green 151A. These mature to yellow in the hot sun, Yellow 11A. Shade leaves are Lime-Green, between Yellow-Green 145A and 144B with red edges and tips, Greyed-Red 181A. Bottom side — Spring, Grey-Brown 199A with veins of Yellow-Green 151A. Summer, Yellow-Green 146D to 145A with areas of Yellow 11A.
Inflorescence:
   Type.—Terminal panicle.
   Number of flowers.—5 to 8.
   Peduncle.—Length — 3.5 cm. Diameter — 1 mm near. Color — Greyed-Purple 185A. Vestiture — Glandular pubescent.
   Pedicel.—Length — 10 to 15 mm. Vestiture — Glabrous. Color — Greyed-Purple 185A.
   Bloom period.—April in Brooks, Oreg.
Flower bud:
   Size.—4 mm wide and 6 mm long.
   Description.—Ovoid.
   Color.—Greyed-Purple 185A.
Flower:
   Type.—Perfect.
   Shape.—Rotate.
   Size.—6 mm deep and 10 mm wide.
   Petal count.—5.
   Petal color.—Pink, Red-Purple 69A.
   Petal description.—Ovate, undulate, 4.5 mm long and 3 mm wide.
   Calyx.—5 sepals, Greyed-Purple 185A, 5 mm long and 3 mm wide.
   Stamen number.—5.
   Stamen color.—Yellow-Green 1A.
   Pollen color.—Yellow 11D.
   Pistil color.—White 155A.
   Fragrance.—None.
Fruit:
   Type.—Two one-seeded samaras, fused at the ovary, shaped like dragonfly wings.
   Color.—Yellow-Green 145A with samaras edged Greyed-Orange 174A.
   Size.—3.5 cm long and 8 mm wide.
Seed:
   Shape.—Ovoid.
   Size.—5 mm long.
   Color.—Brown 200A.
Pest and disease tolerance:

Since Acer shirasawanum ‘MUNN 001’ is a more vigorous grower than Acer shirasawanum ‘Aureum’, it is possible that it is more resistant to the standard Acer diseases like Verticillium wilt. It is unknown at this time. Aphids and scale are known Acer problems. They have not been a problem for this cultivar on the seventeen year old specimen or on the vegetatively propagated material grown in Brooks, Oreg.

### COMPARISONS TO SIMILAR ACER

Compared to Acer shirasawanum ‘Aureum’ (an unpatented plant), this new cultivar grows more than twice as fast. The new plant is easier to propagate from grafts, is more sun tolerant and less gold overall. The new cultivar has chartreuse to lime colored leaves in the summer rather than yellow. The new growth of Acer shirasawanum ‘MUNN 001’ is carmine to red rather than yellow.

I claim:

1. A new and distinct cultivar of Acer shirasawanum plant as shown and described, characterized by its striking carmine to red spring leaves, yellow to lime summer leaves, and vigorous growth rate.
